(19) United States
(12) Patent Application Publication
(10) Pub. No.: US 2019/0393648 A9
(43) Pub. Date: Dec. 26, 2019

(54) DEVICES FOR DETANGLING AND INHIBITING CABLE ENTANGLEMENT DURING MANIPULATION OF CATHETERS

(71) Applicant: St. Jude Medical, Cardiology Division, Inc., St. Paul, MN (US)

(72) Inventors: Israel A. Byrd, Richfield, MN (US); Eric S. Olson, Maplewood, MN (US); Louis-Philippe Richer, Montreal (CA); Chunlan Jiang, Bejing (CN); Kyungmoo Ryu, Palmdale, CA (US); Stuart Rosenberg, Castaic, CA (US); Cyrille Casset, Saint Selve (FR); Hoda Razavi, San Jose, CA (US); Loell B. Moon, Ham Lake, MN (US)

(21) Appl. No.: 15/855,862

(22) Filed: Dec. 27, 2017

Related U.S. Application Data
(60) Provisional application No. 62/472,129, filed on Mar. 16, 2017.
(60) Provisional application No. 62/439,413, filed on Dec. 27, 2016.

(51) Int. Cl. H01R 13/627; A61M 25/01; A61M 39/10; H01R 24/58; H01R 39/02

(52) U.S. Cl. CPC H01R 13/6278; A61M 25/0136; A61M 39/1055; H01R 24/58; A61M 2205/3569; A61M 2039/1022; A61M 2205/10; A61M 2205/3327; H01R 39/02

(57) ABSTRACT

A device to inhibit entanglement of catheter cables comprises a slip ring or a combined slip ring and fluid rotary joint. The device can include a servomechanism configured to power rotation of at least one of the slip ring and the fluid rotary joint. A detangling device for a cable plug configured to connect to a catheter handle comprises an outer cylinder configured to rotate relative to an inner cylinder while electrical connections between the inner and outer cylinders remain intact. A free rotary irrigation channel for a catheter handle inhibits entanglement of irrigation tubing.

### CROSS-REFERENCE TO RELATED APPLICATIONS

This application claims the benefit of U.S. provisional application No. 62/439,413, filed 27 Dec. 2016, and U.S. provisional application No. 62/472,129, filed 16 Mar. 2017, both of which are hereby incorporated by reference in their entirety as though fully set forth herein.

### BACKGROUND

### a. Field

The present disclosure generally relates to medical devices configured for diagnosis or treatment of tissue within a body. In particular, the disclosure relates to devices and mechanisms for inhibiting entanglement and/or detangling electrophysiology (EP) catheter cables.

### b. Background Art

Catheters are used for an ever-growing number of procedures. For example, catheters are used for diagnostic, therapeutic, and ablative procedures, to name just a few examples. Typically, the catheter is manipulated through the patient's vasculature and to the intended site, for example a site within the patient's heart.

A typical EP catheter includes an elongate shaft and one or more electrodes on the distal end of the shaft. The electrodes can be used for ablation, diagnosis, or the like. The shaft is connected to a handle, which a clinician can use to operate and manipulate the catheter.

### BRIEF SUMMARY

A device to inhibit entanglement of catheter cables comprises a slip ring or a combined slip ring and fluid rotary joint. The device can include a servomechanism configured to power rotation of the at least one of the slip ring and the fluid rotary joint. A detangling device for a cable plug configured to connect to a catheter handle comprises an outer cylinder configured to rotate relative to an inner cylinder while electrical connections between the inner and outer cylinders remain intact.

In an embodiment, a rotatable connector for connecting a catheter handle and a cable plug comprises: a first support member configured to be stationary relative to the catheter handle and electrically coupled thereto when the catheter handle is connected to the first support member; and a second support member configured to be stationary relative to the cable plug and electrically coupled thereto when the cable plug is connected to the second support member; wherein the second support member is configured to rotate relative to the first support member when the catheter is rotated with respect to the cable plug.

In another embodiment, a rotatable connector for connecting a catheter handle to a cable plug comprises: at least one of a slip ring and a fluid rotary joint, wherein the at least one of the slip ring and fluid rotary joint is located within at least one of the catheter handle and the cable plug, and wherein the cable plug is configured to be coupled to the catheter handle; and a servomechanism configured to power rotation of the at least one of the slip ring and the fluid rotary joint.

In another embodiment, a detangling device for a cable plug configured to connect to a catheter handle comprises: a first support member comprising a first inner surface and a first outer surface, the first inner surface comprising a plurality of first electrical contacts, the first support member coupled to or located within the catheter handle and configured to be stationary relative to the catheter handle; and a second support member comprising a second inner surface and a second outer surface, the second support member located within the first support member and configured to be stationary relative to the cable plug, wherein the second inner surface of the second support member comprises a plurality of second electrical contacts, each second electrical contact configured to electrically connect to at least one of the first electrical contacts; wherein the cable plug is configured to be inserted into the second support member, and wherein the cable plug and a shaft tip of the catheter are configured to be electrically connected when the cable plug is fully inserted into the second support member; and wherein the first support member is configured to rotate relative to the second support member.

In another embodiment, a rotatable connector for connecting a catheter handle and an irrigation tube comprises: a first support member configured to be stationary relative to the catheter handle; and a second support member configured to be stationary relative to the irrigation tube; wherein the second support member is configured to rotate relative to the first support member when the catheter handle is rotated with respect to the irrigation tube.

### BRIEF DESCRIPTION OF THE DRAWINGS

FIG. 1 is a schematic view depicting a catheter for use in a patient and in association with a cord management system.

FIGS. 2A-2C are high-level block diagrams illustrating various embodiments of a cord management system.

FIG. 3 is a schematic view depicting an embodiment of a detangling mechanism and device.

FIG. 4 is a schematic view depicting another embodiment of a detangling mechanism.

FIG. 5 depicts a partial cross-sectional view along line 5-5 in FIG. 4.

FIG. 6 is a schematic view depicting another embodiment of a detangling mechanism and device.

FIGS. 7A-7F are cross-sectional views depicting various embodiments of engagement/disengagement systems for the detangling mechanism and device shown in, for example, FIG. 6.

FIG. 8 is a schematic view depicting another embodiment of a detangling mechanism and device.

FIGS. 9A-9H are enlarged schematic views depicting various embodiments of portions of FIG. 8.

FIGS. 10A and 10B are schematic views depicting embodiments of a tangle-inhibiting mechanism and device for a catheter.

FIG. 11 is a schematic view depicting another embodiment of a tangle-inhibiting mechanism and device.

FIG. 12 is a schematic view depicting an embodiment of a tangle-inhibiting mechanism and device for use with an irrigated catheter.

FIG. 13 is a schematic view depicting another embodiment of a tangle-inhibiting mechanism and device for use with an irrigated catheter.

FIG. 14 is an exploded view depicting the tangle-inhibiting mechanism and device shown in FIG. 13.

FIG. 15 is a schematic view depicting another embodiment of a tangle-inhibiting mechanism and device for use with an irrigated catheter.

FIGS. 16A and 16B are schematic views depicting embodiments of a tangle-inhibiting mechanism and device for use in conjunction with a servomechanism.

FIG. 17 is an exploded view depicting parts of the tangle-inhibiting mechanism and device, including the servomechanism.

FIGS. 18A-18D are high-level block diagrams illustrating various embodiments of a cord management system.

FIG. 19A is a schematic view depicting another embodiment of a tangle-inhibiting mechanism and device for use with an irrigated catheter.

FIG. 19B is a zoomed in view of a portion of the tangle-inhibiting device shown in FIG. 19A.

FIG. 19C is an exploded in view of a portion of the tangle-inhibiting device shown in FIG. 19A.

FIG. 20 is a schematic view depicting another embodiment of a tangle-inhibiting mechanism and device for use with an irrigated catheter.

FIGS. 21A and 21B are partial cross-sectional views through line 21-21 in FIG. 20.

### DETAILED DESCRIPTION

As a clinician manipulates and rotates a catheter handle, electrical cords and irrigation tubing coming out of the handle can become coiled or tangled. Typically, this requires the clinician to disconnect the cords, untangle them, and then reconnect. To avoid having to perform such a time-consuming and frustrating task in the middle of an EP procedure, clinicians need a cord management system, such as a device for detangling electrical cords and/or irrigation tubing and/or a device for inhibiting or preventing tangling of electrical cords and/or irrigation tubing.

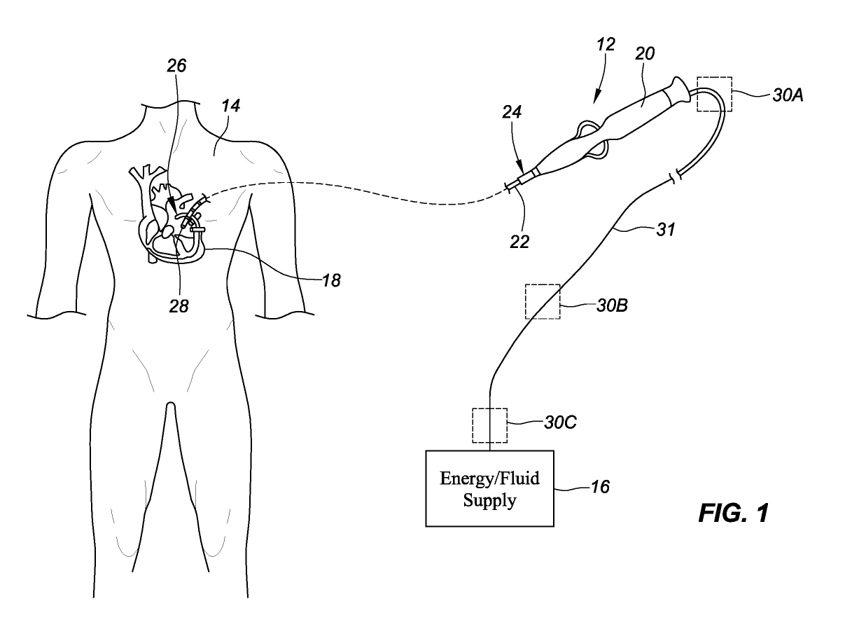

FIG. 1 is a schematic view depicting a therapeutic or diagnostic catheter 12 in use in a patient's body 14 and connected to an energy/fluid supply 16 (e.g., an RF ablation generator) according to the present disclosure. In an embodiment, the catheter 12 may be an ablation catheter. The catheter 12 can be configured to be inserted into a the patient's heart 18. The catheter 12 may include a handle 20 and a shaft 22 having a proximal end portion 24, a distal end portion 26, and a tip portion 28 disposed at the distal end portion 26 of the shaft 22. The catheter 12 may further include other conventional components such as, for example and without limitation, a temperature sensor, a position sensor, additional sensors or electrodes, and corresponding conductors or leads.

The shaft 22 can be an elongate, tubular, flexible member configured for movement within the body 14. The tip portion 28 of the shaft 22 supports, for example and without limitation, sensors and/or electrodes mounted thereon. The tip portion 28 may include ablation elements (e.g., ablation tip electrodes for delivering RF ablative energy). The shaft 22 may also permit transport, delivery, and/or removal of fluids (including irrigation fluids, cryogenic ablation fluids, and bodily fluids), medicines, and/or surgical tools or instruments.

Various embodiments and locations of a cord manager 30A, 30B, 30C are shown in phantom in FIG. 1. The cord manager can inhibit tangling and/or detangle one or more electrical cords and/or irrigation tubing 31 (shown in FIG. 1 as a single “cord” for simplicity) connecting the catheter handle 20 to the energy/fluid supply 16. In one embodiment, the cord manager 30A can be attached to or comprise part of the catheter handle 20. In another embodiment, the cord manager 30B can be independent from (and physically separated from) both the catheter handle 20 and the energy/fluid supply 16. In a third embodiment, the cord manager 30C can be attached to or comprise part of the energy/fluid supply 16.

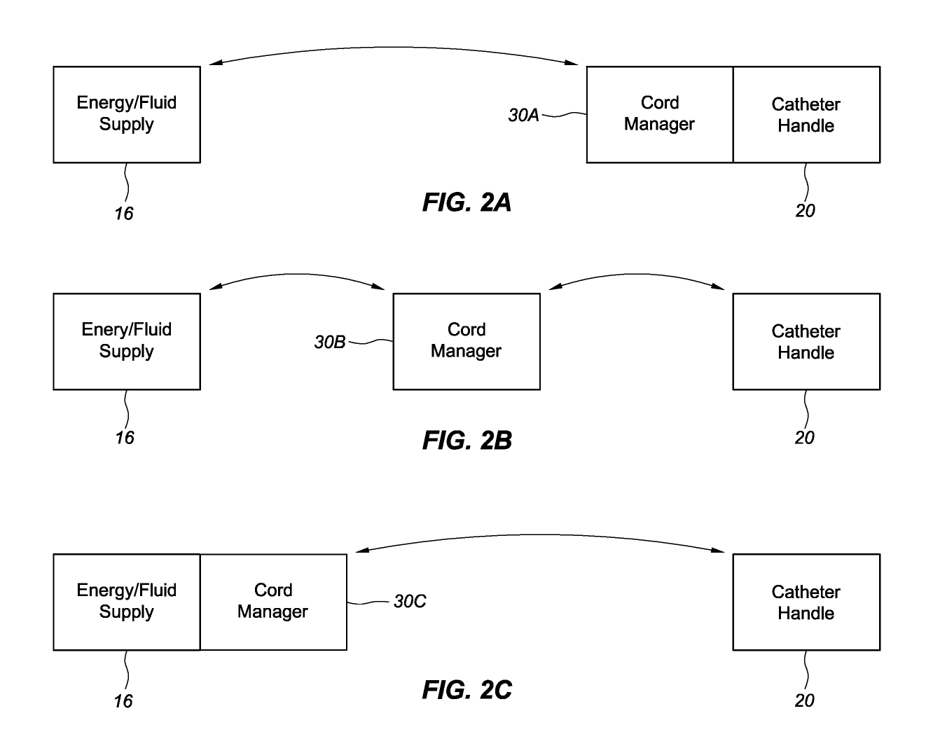

FIGS. 2A-2C provide high-level block diagrams illustrating various possible relationships between components of a cord management system. In FIG. 2A, the cord manager 30A is attached to or comprises part of the catheter handle 20 and is configured to be connected (via a plurality of cords) to the energy/fluid supply 16. An example of a cord manager includes an electrical slip ring, described in further detail below. Electrical slip rings are devices used in a variety of industries to allow the transmission of power and electrical signals from a stationary structure to a rotating structure. In FIG. 2B, the cord manager 30B is independent from both the catheter handle 20 and the energy/fluid supply 16, but is configured to be functionally connected to both. In FIG. 2C, the cord manager 30C is attached to or comprises part of the energy/fluid supply 16 and is configured to be connected to the electrical cords and/or irrigation tubing connected to the catheter handle 20.

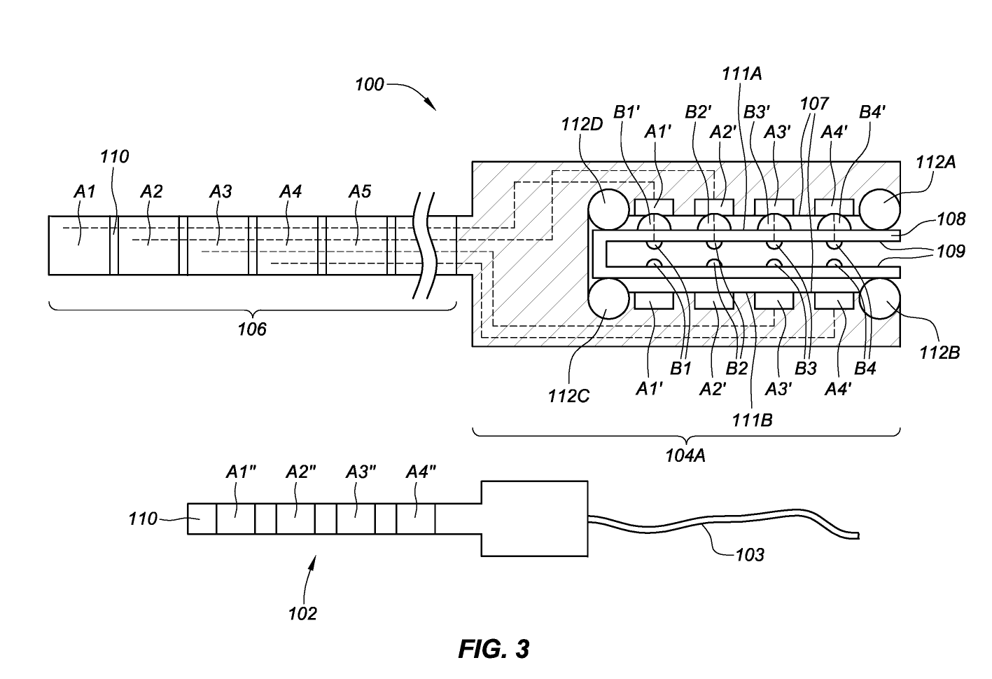

FIG. 3 illustrates an example of one type of cord management system, a detangling mechanism and device, in accordance with the present disclosure. The two main components of the detangling mechanism and device are a catheter 100 and a tail cord plug 102. A cord portion 103 of the tail cord plug 102 can connect to a radio frequency generator, an EP recording system, or an electro-anatomical localization and visualization system (e.g., such as the ENSITE system of St. Jude Medical, Inc.), for example. The catheter 100 can be a diagnostic or therapeutic EP catheter, for example. The catheter comprises a handle 104A and a shaft tip 106. Within the handle 104A is a tail cord plug receptacle 108, which is configured to receive the tail cord plug 102.

The shaft tip 106 can be made of a biocompatible polymeric material 110, such as polytetrafluoroethylene (PTFE) tubing (e.g., TEFLON® brand tubing) or other polymeric materials or thermoplastics, such as polyamide-based thermoplastic elastomers (namely poly(ether-block-amide), such as PEBAX®). The shaft tip 106 includes a plurality of ring electrodes A1-A5 located on an outer surface of the shaft tip 106. The ring electrodes A1-A5 can be directly and individually wire-connected to band electrodes A1′-A4′ on the inner surface 107 of the handle 104A. Alternatively, the electrodes A1-A5 can be wire-connected in series to band electrodes A1′-A4′ on the inner surface 107 of the handle 104A. Each ring electrode A1-A3 can be connected to its corresponding band electrode A1′-A3′, respectively. In this example, the ring electrodes A4 and A5 can both be connected to a single corresponding band electrode A4′. Thus, more than one ring electrode on the shaft tip 106 can be connected to a single electrode on the inner surface 107 of the handle 104A. Similarly, one electrode on the shaft tip 106 can be connected to more than one electrode on the inner surface 107 of the handle 104A.

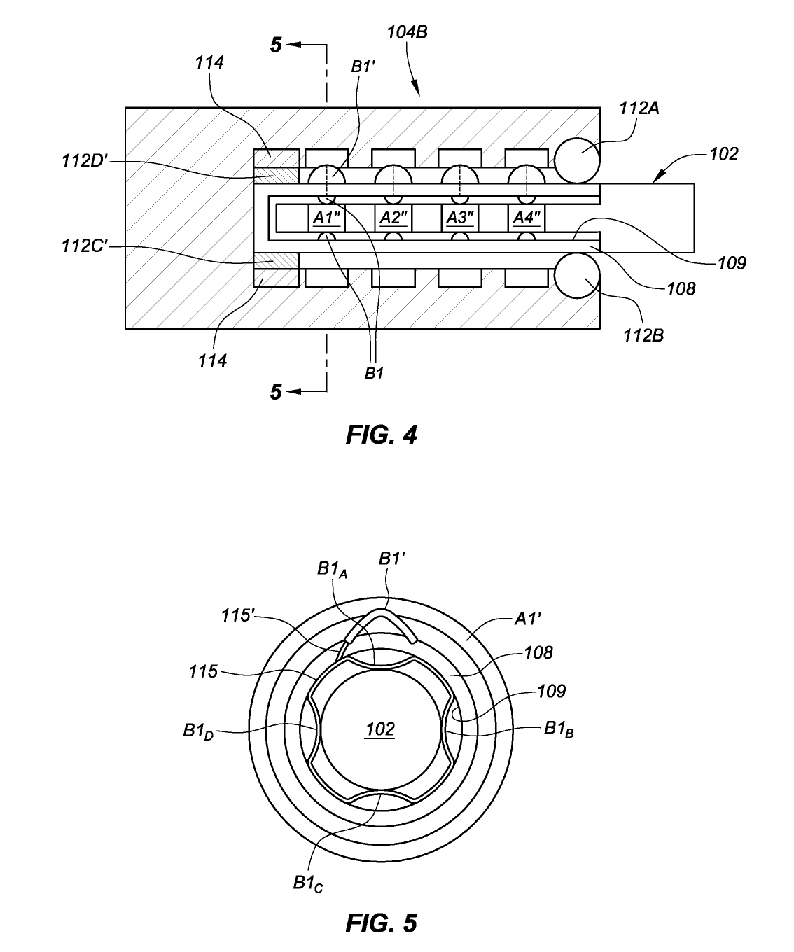

The handle 104A can further comprise four sets of electrical contacts, such as pin electrodes B1-B4. (In other embodiments the electrical contacts can be brushes.) Each set of pin electrodes B1-B4 can comprise 4 separate pin electrodes that can be located 90 degrees apart from one another on the inner surface 109 of the tail cord plug receptacle 108. In some embodiments, each set of pin electrodes B1-B4 can comprise three pin electrodes. Each of the pin electrodes in the set of pin electrodes B1 is wire-connected, both to the other B1 pin electrodes (designated B1_{A-D} in FIG. 5) and to their counterpart pin electrode B1′ on an outer surface 111A of the tail cord plug receptacle 108. Similarly, each of the pin electrodes in each set of pin electrodes B2-B4 are wire-connected, both to the other pin electrodes in each set of pin electrodes B2-B4, respectively, and to their counterpart pin electrode B2′-B4′, respectively, on the outer surface 111A of the tail cord plug receptacle 108. The tail cord plug 102 comprises ring electrodes A1″-A4″, which each electrically couples to one of the set of pin electrodes B1-B4, respectively, when the tail cord plug 102 is fully inserted into the tail cord plug receptacle 108. Therefore, when the tail cord plug 102 is fully inserted into the tail cord plug receptacle 108, a secure series of electrical connections links the catheter shaft tip 106 to the tail cord plug 102. In addition, the catheter handle 104A can rotate around the tail cord plug receptacle 108 without any interruption in the series of electrical connections. To allow for smooth rotation of the handle 104A around the tail cord plug receptacle 108, a plurality of ball bearings 112A-D or roller bearings 112C′ and 112D′ (see FIG. 4) can be placed between the inner surface 107 of the handle 104A and the outer surface 111A, 111B of the tail cord plug receptacle 108, as shown.

FIG. 4 illustrates a catheter handle 104B, which is an alternative embodiment of the catheter handle 104A shown in FIG. 3. In this embodiment, the tail cord plug 102 (shown here without cord portion 103 for simplicity) is fully inserted into the tail cord plug receptacle 108 of the handle 104B. In place of the ball bearings 112C and 112D shown in FIG. 3, the present embodiment includes roller bearings 112C′ and 112D′. These roller bearings serve the same function as the ball bearings (i.e., to reduce friction and to facilitate rotation of the catheter handle 104B relative to the tail cord plug receptacle 108). In this example, the roller bearings 112C′ and 112D′ each sit in a race 114 in which the roller bearing slides or rolls. Although not shown, the ball bearings 112A-112D may also sit in races.

FIG. 5 shows a partial cross-sectional view through line 5-5 in FIG. 4. As illustrated, the pin electrodes B1_{A}, B1_{B}, B1_{c}, and B1_{D} are connected both to each other and to counterpart pin electrode B1′. The pin electrodes B1_{A}, B1_{B}, B1_{c}, and B1_{D} can be connected via a flexible unitary element, such as a wire 115 with an extending portion 115′. It should be noted that the counterpart pin electrode B1′ comes to a domed point in order to reduce contact and friction between the counterpart pin electrode B1′ and the band electrode A1′. In other embodiments, the pin electrode B1′ may have other shapes, such as a triangular shape, for example.

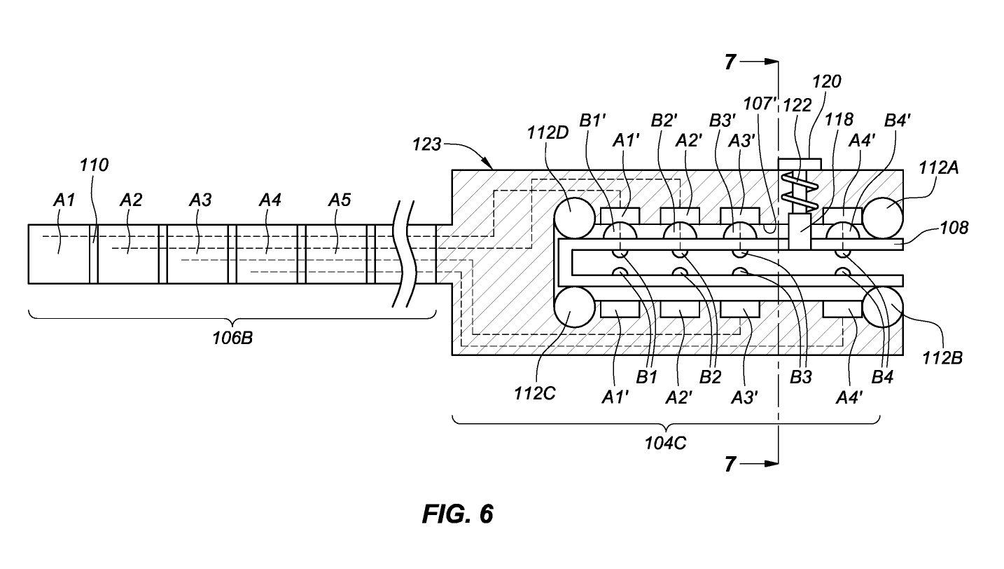

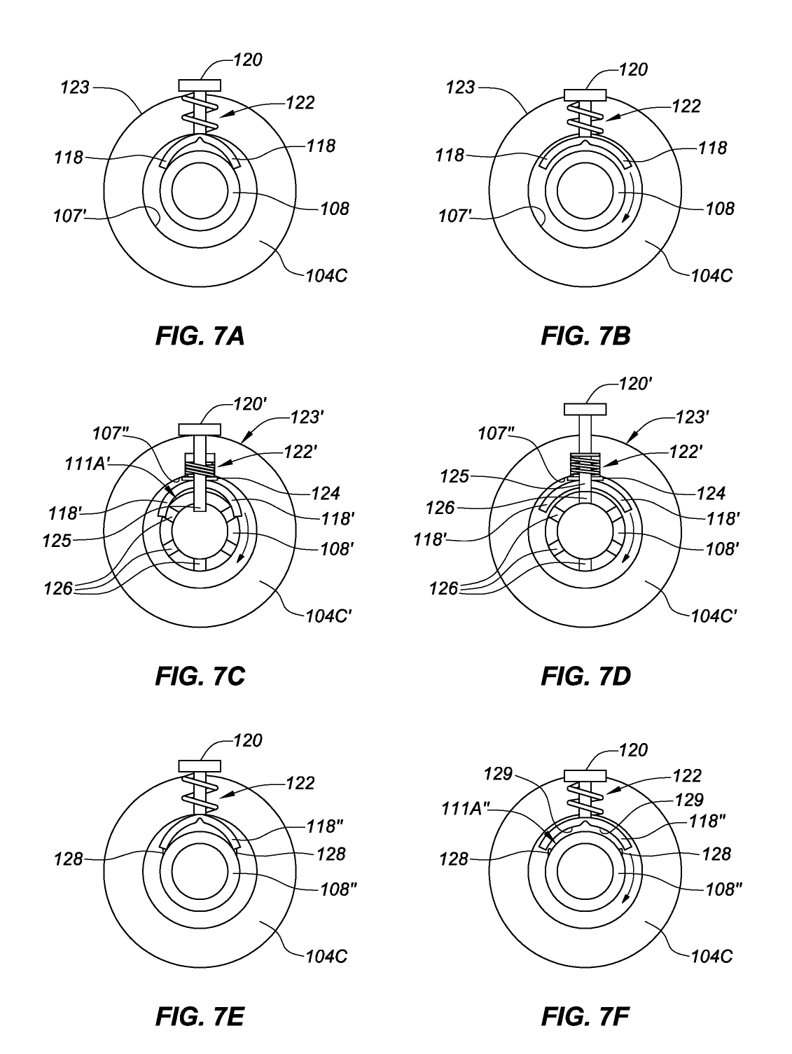

FIG. 6 depicts another embodiment of a portion of the detangling mechanism and device shown in FIG. 3. This embodiment includes an engagement/disengagement system that can be used to reduce the electromechanical noise potentially caused by detangling, thereby reducing the likelihood that the detangling process would interfere with mapping or ablation signals, for example. In particular, the engagement/disengagement system depicted in the embodiment of FIG. 6 is a ratchet-like mechanical system including a pair of clam arms 118 (shown in FIGS. 7A-F) and a detangle button 120 connected to a spring structure 122 (the spring and the rod from the underside of the detangle button 120 to the outer surface of the clam arms 118) that can be used to lock and unlock the clam arms 118. The spring structure 122 can be embedded within the handle 104C. The detangle button 120, which is connected to the spring structure 122, can protrude above the outer surface 123 of the handle 104C. The clam arms 118 are also connected to the spring structure 122, and can protrude from the upper inner surface 107′ of the handle 104C and can be configured to engage the tail cord plug receptacle 108.

FIGS. 7A-7F are partial cross-sectional views of various embodiments of the ratchet-like mechanical engagement/disengagement system described above with respect to FIG. 6. FIGS. 7A and 7B illustrate a first embodiment of the ratchet-like system. In FIG. 7A, the system is in its default locked state. In the locked state, the detangle button 120 is in its default position (i.e., not pressed down), the spring structure 122 is in its non-compressed state, and the clam arms 118 are engaging the tail cord plug receptacle 108 via friction, so as to inhibit the tail cord plug receptacle from rotating relative to the handle 104C. FIG. 7B shows the unlocked position of this embodiment, in which the detangle button 120 has been pressed down, the spring structure 122 is compressed, and the clam arms 118 lift outwards relative to the longitudinal axis of the handle 104C, away from the tail cord plug receptacle 108. Because the clam arms 118 are no longer able to engage the tail cord plug receptacle 108, the tail cord plug receptacle 108 is free to rotate relative to the remainder of the handle 104C, thereby allowing for detangling of the cord portion 103.

FIGS. 7C and 7D illustrate a second embodiment of the ratchet-like system. In FIG. 7C, showing the default locked state, a detangle button 120′ sits atop the outer surface 123′ of the handle 104C′. The spring structure 122′ is compressed against an annular ledge 124 proximate the upper inner surface 107″ of the handle 104C′. In this embodiment, the spring structure 122′ is connected to a locking pin 125 that is configured to fit into one of a plurality of locking pin holes 126 through the tail cord plug receptacle 108′. In the locked state, the locking pin descends into a locking pin hole 126 to inhibit rotation of the tail cord plug receptacle 108′ relative to the handle 104C′. In addition, in the FIG. 7C configuration, the clam arms 118′, which are optional in this embodiment, engage the outer surface 111A′ of the tail cord plug receptacle 108′ and provide a frictional force to further inhibit rotation of the tail cord plug receptacle 108′ relative to the handle 104′.

In FIG. 7D, the detangle button 120′ has been pulled up to overcome the spring force otherwise pulling the spring into its compressed state and to thereby unlock the ratchet-like system. When the detangle button 120′ is pulled up, the spring structure 122′ expands and the locking pin 125 is raised so that it is no longer inserted into one of the locking pin holes 126. In addition, the clam arms 118′ lift outwards, away from the tail cord plug receptacle 108′. Because the locking pin 125 is released from the locking pin holes 126 and the clam arms 118′ are no longer able to engage the tail cord plug receptacle 108′, the tail cord plug receptacle 108′ is free to rotate relative to the handle 104C′, thereby allowing for detangling of the cord portion 103.

FIGS. 7E and 7F illustrate a third embodiment of the ratchet-like system. This embodiment is similar to the first embodiment described above with respect to FIGS. 7A and 7B, with the additional features of locking pins or spikes 128 on the outer surface 111A″ of the tail cord plug receptacle 108″ and, optionally, corresponding serrations or notches (not shown) on the inner surface 129 of the clam arms 118″. In the locked state shown in FIG. 7E, the locking pins or spikes 128 provide further resistance to rotational movement of the tail cord plug receptacle 108″, in addition to the frictional resistance provided by of the clam arms 118″.

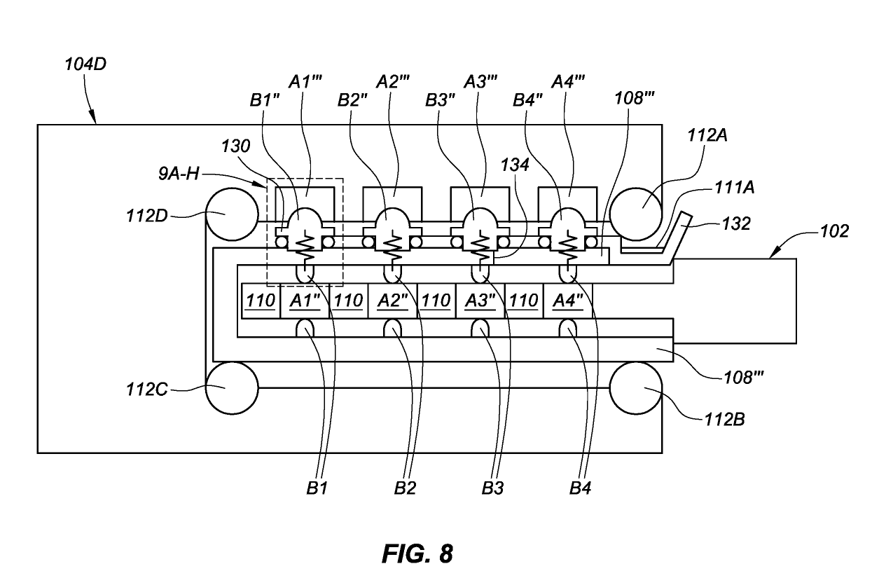

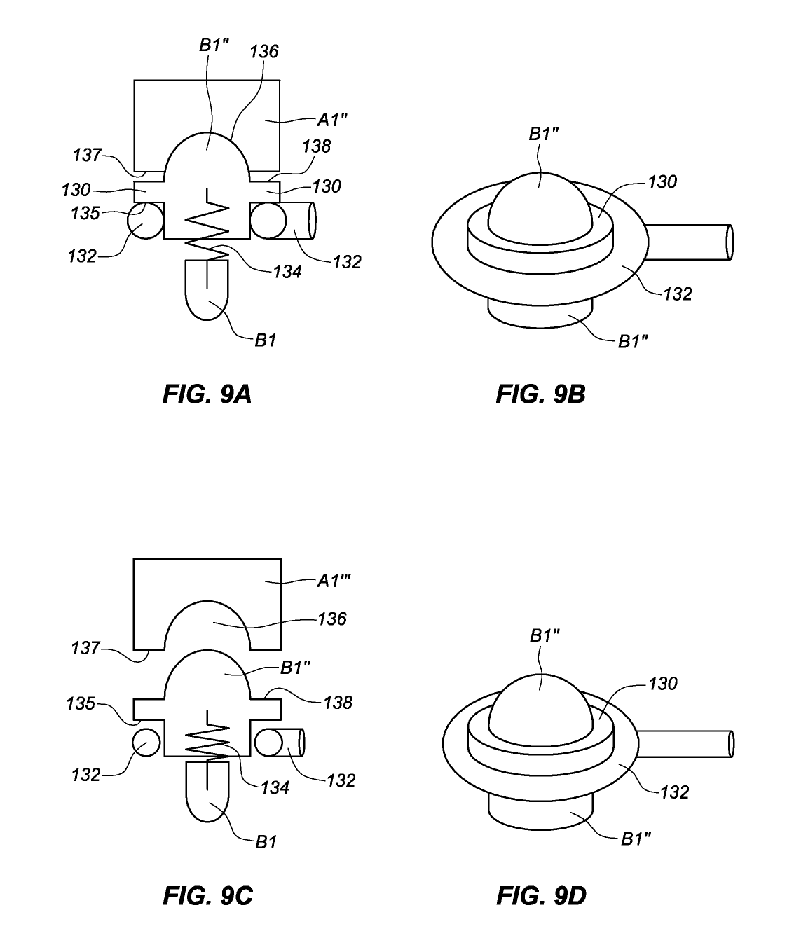

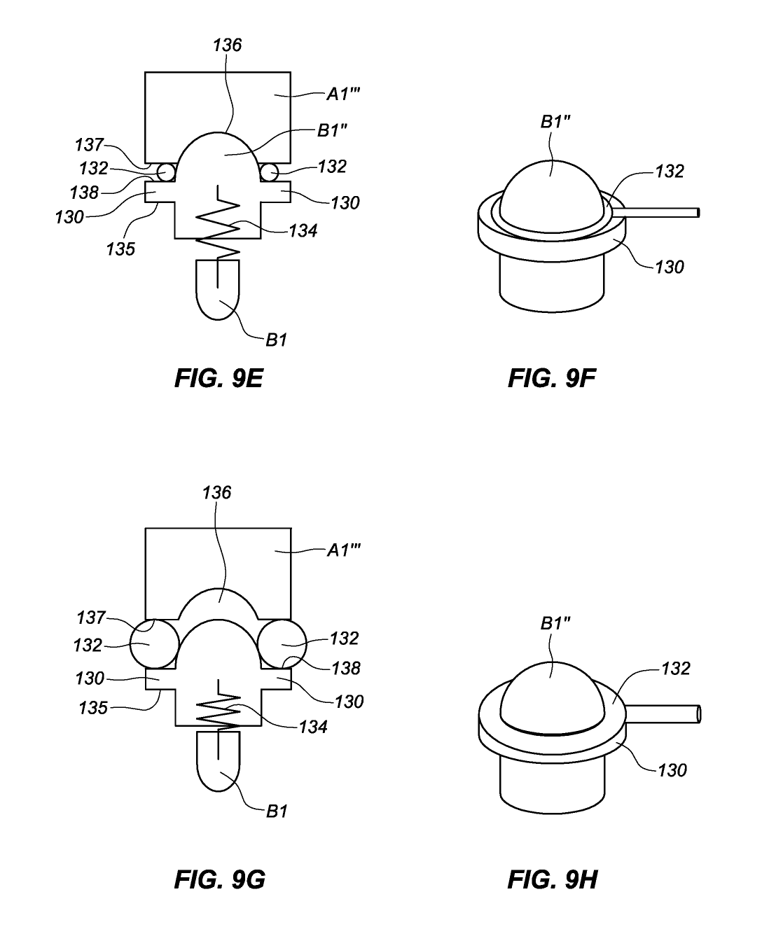

FIG. 8 depicts another embodiment of a portion of the detangling mechanism and device shown in FIG. 3, including another embodiment of an engagement/disengagement system that can be used to reduce the electromechanical noise potentially caused by detangling. In particular, the engagement/disengagement system depicted in the embodiment of FIG. 8 is an inflation/deflation system. In this embodiment, the ring electrodes A1′-A4′ shown in FIG. 3 have been replaced with pocket electrodes A1′″-A4′″. The pocket electrodes A1′″-A4′″ can be connected, individually or in series, via electrical wires to the ring electrodes A1-A4 (see FIG. 3). The pocket electrodes A1′″-A4′″ are configured to receive pin electrodes B1″-B4″, respectively. The pin electrodes B1″-B4″ are shaped differently from the pin electrodes B1′-B4′ shown in FIG. 3. For example, each of the pin electrodes B1″-B4″ includes an annular edge 130 with a diameter corresponding to that of the electrode pockets A1′″-A4′″. The annular edges 130 can rest upon a pneumatic support structure 132 comprising an inflation valve located between the annular edges 130 and an outer surface of the tail cord plug receptacle 108′″ (see FIG. 8). In an alternative embodiment, the pneumatic support structure 132 can be located between the annular edges 130 and the electrode pockets A1′″-A4′″ (see FIGS. 9E-9H). In either case, the pneumatic support structure 132 can form a network of linked donut-shaped balloons around each of pin electrodes B1″-B4″. The pin electrodes B1″-B4″ can be electrically wired to the pin electrodes B1-B4, and can also be connected via a mechanical spring 134, which, together with the inflation/deflation system, allow for engagement and disengagement of the detangling device.

FIGS. 9A-9H illustrate enlarged versions of various embodiments of pocket electrode A1″, pin electrode B1″ and pin electrode B1, all shown in the dotted-line rectangle labeled 9A-H in FIG. 8. Although not shown, similar embodiments can exist for pocket electrodes A2′″-A4′″, pin electrodes B2″-B4″, and pin electrodes B2-B4. FIGS. 9A-9D illustrate a first embodiment in which the pneumatic support structure 132 is located between the bottom surface 135 of the annular edge 130 of pin electrode B1″ and the outer surface 111A of the tail cord plug receptacle 108′″ (see FIG. 6). FIG. 9A is a side view and FIG. 9B is an isometric top and side view, both showing a locked position in which the pneumatic support structure 132 is inflated. The pneumatic support structure 132 can be inflated, for example, via a luer-lock syringe. When inflated, the pneumatic support structure 132 pushes up on the bottom surface 135 of annular edge 130, forcing pin electrode B1″ into the pocket portion 136 of pocket electrode A1″. In this position, the spring 134 is stretched (i.e., the spring 134 is attached to both B1 and B1″ and is attempting to pull these components together). When pin electrode B1″ is engaged with the pocket portion 136 of pocket electrode A1″ and the spring 134 is stretched, the tail cord plug receptacle 108′″ is inhibited from rotating relative to the catheter handle 104D. When the pneumatic support structure 132 is deflated, as shown in FIGS. 9C and 9D, it no longer pushes up on the bottom surface 135 of annular edge 130 and pin electrode B1″ is no longer engaged with the pocket portion 136 of pocket electrode A1′″. In this state, the spring 134 is compressed (default state (as used herein, the term “default” state means the state or condition when no external force is being applied)), allowing the top of pin electrode B1″ to clear the bottom surface 137 of pocket electrode A1′″, which, in turn, allows for rotation of the tail cord plug receptacle 108′″ relative to the catheter handle 104D. Detangling can take place in this unlocked state.

FIGS. 9E-9H illustrate a second embodiment in which the pneumatic support structure 132 is located between the top surface 138 of the annular edge 130 of pin electrode B1″ and the bottom surface 137 of pocket electrode A1′″. FIG. 9E is a side view and FIG. 9F is an isometric top and side view, both showing a locked position in which the pneumatic support structure 132 is deflated and the spring 134 is stretched (default state). In this locked state, pin electrode B1″ is engaged with the pocket portion 136 of pocket electrode A1′″, thereby inhibiting rotation of the tail cord plug receptacle 108′″ relative to the catheter handle 104D. When the pneumatic support structure 132 is inflated, as shown in FIGS. 9G and 9H, it causes pin electrode B1″ to descend out of the pocket portion 136, allowing the top of pin electrode B1″ to clear the bottom surface 137 of pocket electrode A1′″. This, in turn, allows for rotation of the tail cord plug receptacle 108′″ relative to the catheter handle 104D, thereby unlocking the detangling device.

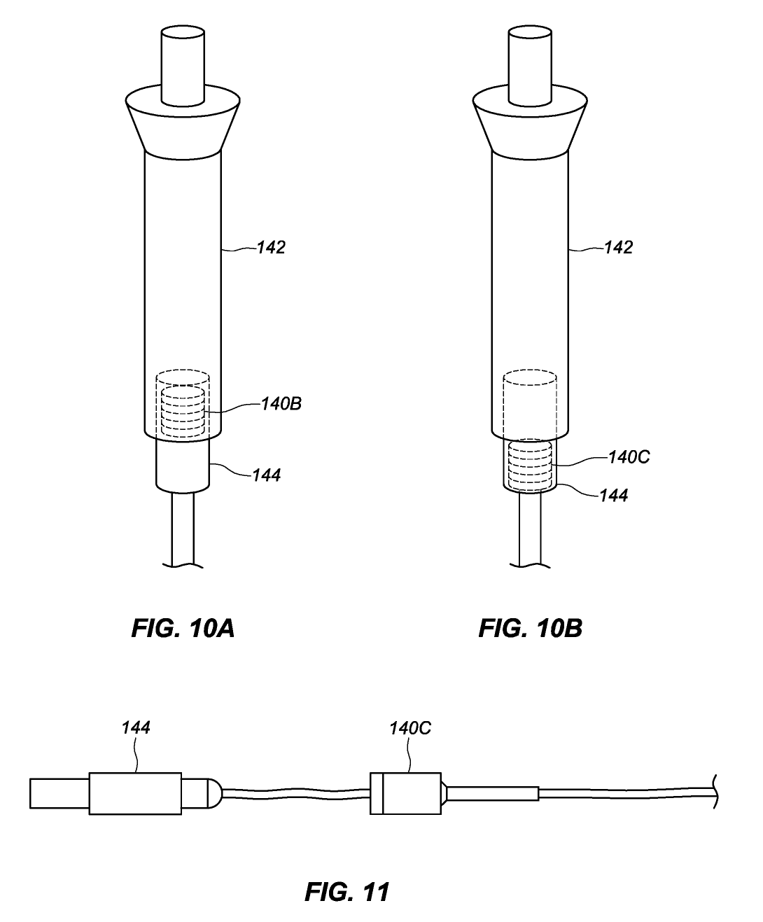

FIG. 10A depicts another example of a cord management system, an electrical slip ring 140B, coupled with a catheter handle 142 in an arrangement similar to that discussed above with respect to FIG. 2A. In this example, the slip ring 140B is placed inside of the catheter handle 142. FIG. 10B depicts a slip ring 140C coupled with a connector 144 (e.g., a connector linking a slip ring with an energy/fluid source), similar to the arrangement described above with respect to FIG. 2C. In this example, the slip ring 140C is placed inside of the connector 144. FIG. 11 is another depiction of the slip ring 140C coupled with the connector 144. In this example, connector housing (not shown) can be modified to include the bearings, brushes, and other components required to allow the slip ring 140C to be built into the connector 144. The cable on either side of the slip ring 140C may be joined to the slip ring 140C via soldering or press fitting, for example.

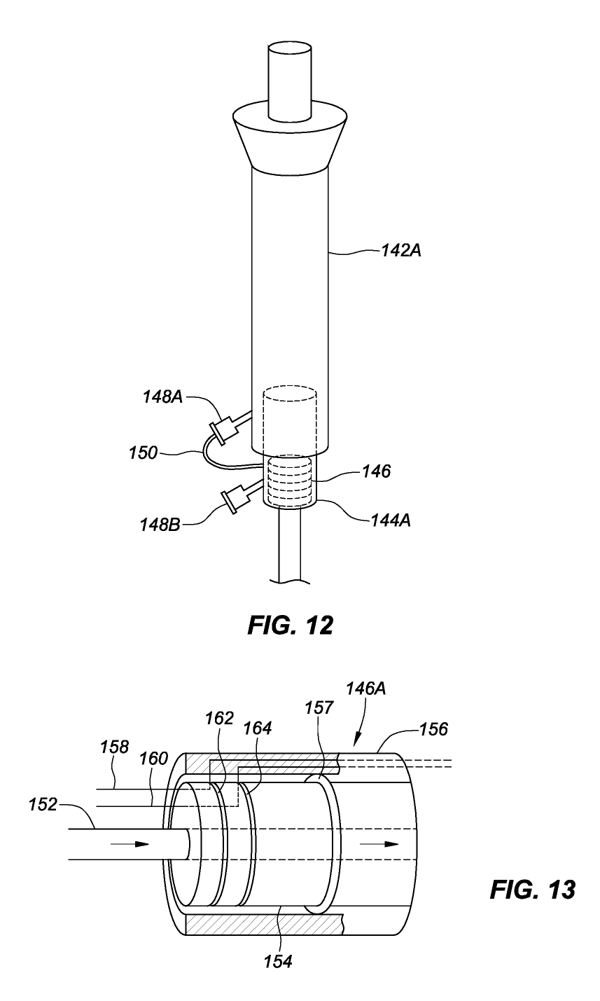

FIG. 12 illustrates an embodiment of a hybrid electrical slip ring and fluid rotary joint 146 configured for use with a handle of an irrigated catheter 142A. In particular, the hybrid slip ring and fluid rotary joint 146 allows the handle of irrigated catheter 142A to rotate without rotating the electrical cables or fluid channel. In this “retrofit” embodiment, a luer lock irrigation port 148A is connected to the handle 142A. A luer-lock-compatible fluid bypass tube 150 connects irrigation port 148A to connector the hybrid slip ring and fluid rotary joint 146. As a connector 144A rotates with respect to the handle 142A, fluid from the hybrid slip ring and fluid rotary joint 146 is able to pass into the handle 142A without rotation of the bypass tube 150. In an another embodiment, another luer lock irrigation port 148B can be attached to the connector 144A and luer lock-compatible fluid bypass tubing can be used to connect this irrigation port to the handle 142A. In another embodiment, luer lock irrigation ports can be attached to both the handle 142A (as shown) and to the connector 144A.

FIG. 13 illustrates another embodiment of a hybrid electrical slip ring and fluid rotary joint 146A configured for use with an irrigated catheter. In this embodiment, irrigant flows through a fluid lumen 152 distally away from the catheter handle (not shown). The fluid lumen 152 runs through the center of the hybrid electrical slip ring and fluid rotary joint 146A. The hybrid electrical slip ring and fluid rotary joint 146A also includes an inner support member, such as inner barrel 154, which rotates relative to an outer support member, such as outer barrel 156, at an o-ring 157. (In other embodiments, the inner and outer support members can include parallel plates.) The outer barrel 156 can be part of or connected to the handle (e.g., handle 142 in FIGS. 10A and 10B). Two electrical wires 158 and 160 make contact with electrical slip rings 162 and 164, respectively, located around the circumference of the inner barrel 154. In other embodiments, more than two electrical pathways may be used.

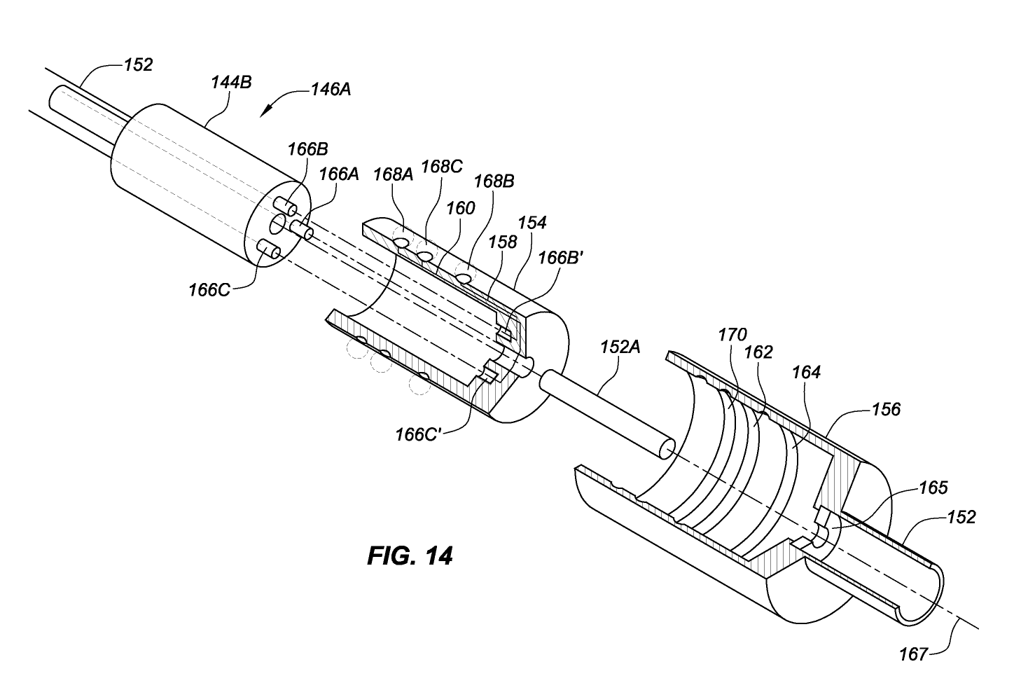

FIG. 14 is an exploded, isometric view of the hybrid electrical slip ring and fluid rotary joint 146A shown in FIG. 12. In this embodiment, the fluid lumen 152 includes a smaller diameter section 152A (running through the connector 144B, the inner barrel 154, and the outer barrel 156) that is connected to the fluid lumen 152 via an o-ring 165. In this embodiment, the connector 144B has three pins 166A, 166B, and 166C, located circumferentially around the central axis 167 of both the fluid lumen 152 and the hybrid electrical slip ring and fluid rotary joint 146A. In this embodiment, pins 166A, 166B, and 166C are each separated by about 120 degrees. Although only three pins are shown in this embodiment, the connector 144B can include up to about 128 pins. The pins 166B and 166C are received by pin receptacles 166B′ and 166C′, respectively, in the inner barrel 154. The pin receptacle that receives the pin 166A is not shown in FIG. 14. The pin receptacles 166B′ and 166C′ can be connected to electrical wires—in this case, to the wires 158 and 160, respectively. The electrical wires 158 and 160, in turn, are connected to conductive ball bearings 168B and 168C. The ball bearing 168A can be connected to the receptacle (not shown) that receives the pin 166A. The ball bearings 168A, 168B, and 168C can be positioned within conductive troughs forming slip rings 170, 164, and 162, respectively, and configured to rotate within these troughs as the inner barrel 154 rotates relative to the outer barrel 156. The ball bearings provide for a smooth mechanical rotation, as well as electrical connections between the pins and slip rings.

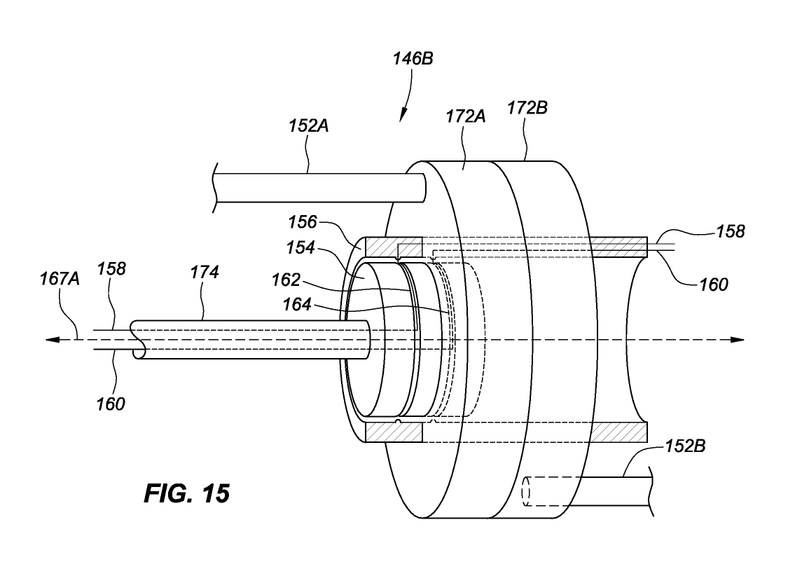

FIG. 15 shows another embodiment of a hybrid electrical slip ring and fluid rotary joint 146B. In this embodiment, the toroid has two halves, 172A and 172B. As shown in FIG. 15, the left half of the toroid 172A rotates relative to the right half of the toroid 172B. The central lumen 174 holds the electrical wires 158 and 160, and the fluid lumen, including the parts 152A and 152B, runs parallel to the central lumen 174. In this embodiment, the two parts of the fluid lumen 152A and 152B do not line up (although it is possible that they may temporarily line up during use); instead, fluid flows from one part to another via a channel that travels through the two toroid halves, 172A and 172B. The channel can have a variety of shapes and must be water tight to the exterior of the system.

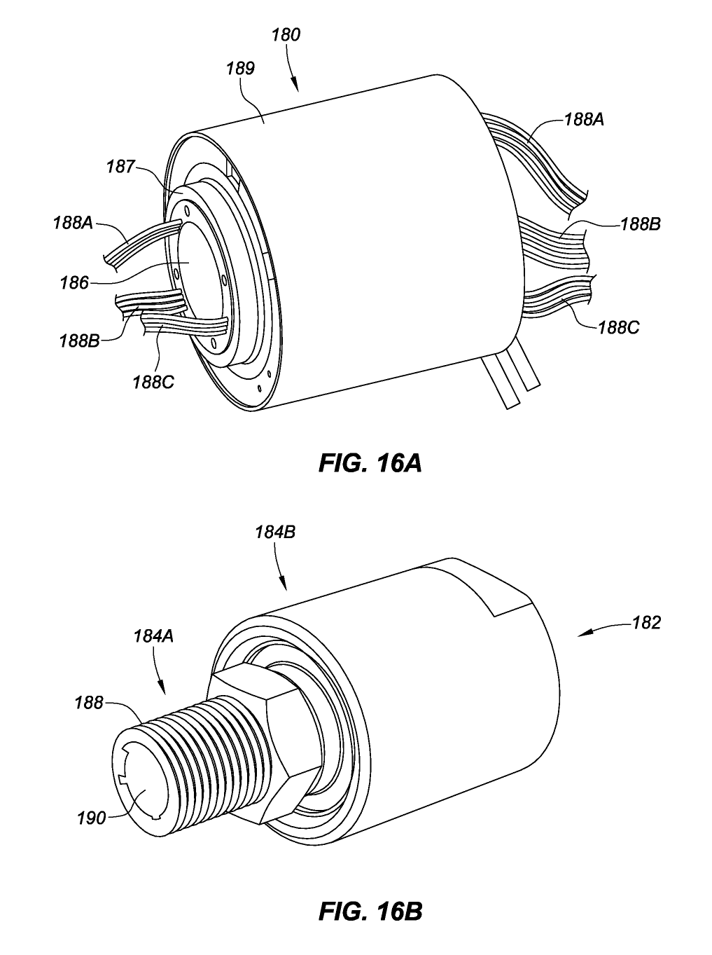

FIGS. 16A and 16B illustrate examples of an electrical slip ring 180 and a fluid rotary joint 182 that can be assembled together to provide part of a servomechanism to mitigate catheter cord tangling, as further described below with respect to FIG. 17. In particular, the threaded portion 184A of fluid rotary joint 182 fits into the central bore 186 of the electrical slip ring 180. An inner barrel 187 of the electrical slip ring 180, along with its associated groups of electrical wires 188A, 188B, and 188C, rotates relative to a rotating outer barrel 189 of the electrical slip ring 180. Similarly, the inner threaded portion 184A of the fluid rotary joint rotates relative to a rotating outer barrel 184B. The inner portions 187 and 184A are stationary relative to one another, and the outer portions 189 and 184B are stationary to one another. Fluid flows through the central lumen 190 of the fluid rotary joint 182.

FIG. 17 is a stylized, exploded view of parts of another embodiment of a cord management system, including a servomechanism 300. The servomechanism 300 can be held stationary, such as by being clamped to a patient bed rail or table. A catheter 192 may include a luer lock 194, onto which may be attached (e.g., as a retrofit) a motion processing unit 196, such as an accelerometer (and/or gyroscope and/or inclinometer). The motion processing unit 196, which is commercially very small and likely not visible, may also be placed in other locations, such as a connector cable or the catheter handle, for example. The motion processing unit 196 is configured to sense rotation of the catheter handle 192, which is similar to the catheter handle 20 shown in FIG. 1 (minus the cords 31). As the catheter handle 192 rotates, the inner barrel of the electrical slip ring 187 rotates to match the catheter handle 192 angle of rotation. Nevertheless, it is not possible for the rotation of the catheter handle 192 to impart enough torque to the combined slip ring/rotary joint 180/182 through a flexible extension cable and fluid tubing (due to the high torque of the fluid rotary joint 182). Therefore, rotation of the combined slip ring/rotary joint 180/182 must be provided by another means. This means can be a powered servomotor 198 connected to the inner and outer barrels 187, 189 of the slip ring 180 via a timing pulley 200 and timing belt. Alternatively, other means of turning the slip ring/rotary joint 180/182 may be provided, such as a direct drive.

The servomotor 198 can be configured to communicate with the accelerometer 196 via a wired (e.g., an extension cable) or wireless connection. The rotation angle of the servomotor 198 and thus the slip ring/rotary joint 180/182 can be determined from the rotation angle of the catheter handle 192 (as determined by the accelerometer 196) to inhibit or prevent tangling. In an embodiment, signals output by the accelerometer 196 can be used to determine the roll angle of the catheter handle 192, which, in turn, can be communicated to the servomotor 198 to effect the necessary rotation of the slip ring/rotary joint 180/182. Determination of the roll angle of the catheter handle 192 can be made via various means known in the art, including a magnetic location system or an optical location system.

In an embodiment, the roll angle of the catheter handle can be determined from the following equation:

$$roll = β = tan ( \frac{{A}_{y}}{\sqrt{{( {A}_{x} )}^{2} + {( {A}_{z} )}^{2}}} )$$

where the each of the ‘A’ values are the calibrated/normalized values of the accelerometer output and where the y-axis of the accelerometer is aligned with the longitudinal axis of the catheter handle.

The control system can be implemented such that the servomotor 198 attempts to keep the roll angle of the catheter handle 192 the same as the angle of the slip ring/rotary joint 180/182 at all times to within some tolerance (e.g., 1 degree).

Alternatively, the system could count the number of full turns (rolls) of the catheter handle 192 and only rotate the slip ring/rotary joint 180/182 when a full 360 degrees of rolls has been achieved. Because the cabling is not sensitive to a small amount of wrapping, this may be entirely acceptable. This magnitude can also be user-settable. In an example, the system can rotate the slip ring/rotary joint 180/182 on each half rotation (180 degrees) of the catheter handle 192. Limiting when the system rotates the slip ring/rotary joint 180/182 can be beneficial in order to minimize any audible or electrical noise caused by the servomotor 198.

Determination of the roll angle of the catheter handle 192 may also be made via other means. For example, the roll angle may be determined using a magnetic location system with a six-degrees-of-freedom sensor embedded in the catheter handle 192. The MediGuide system owned by St. Jude Medical, Inc. provides such capability. In another embodiment, the roll angle may be determined by sensing the location of the catheter handle 192 optically or through a vision system.

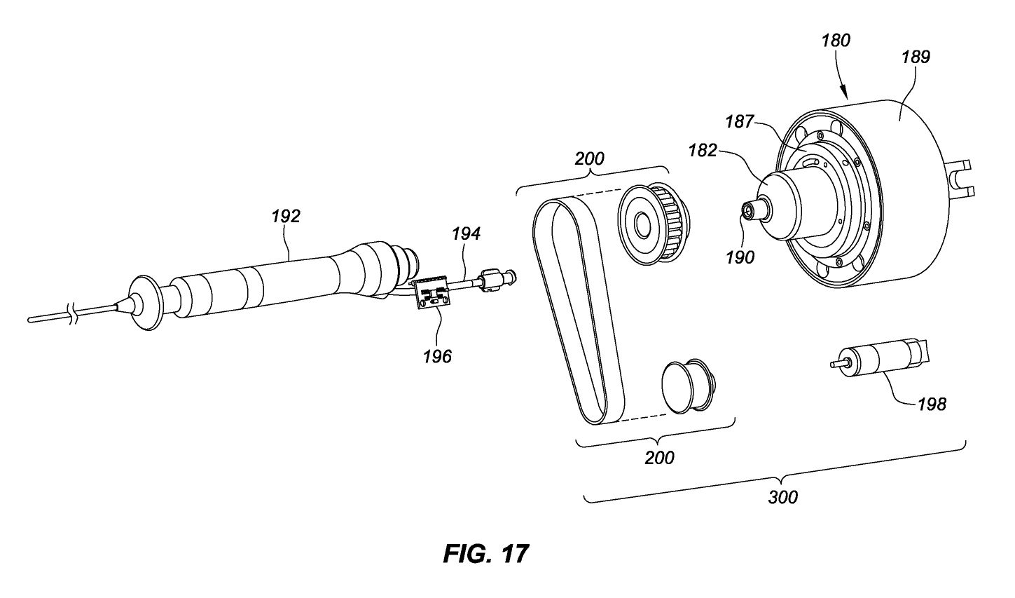

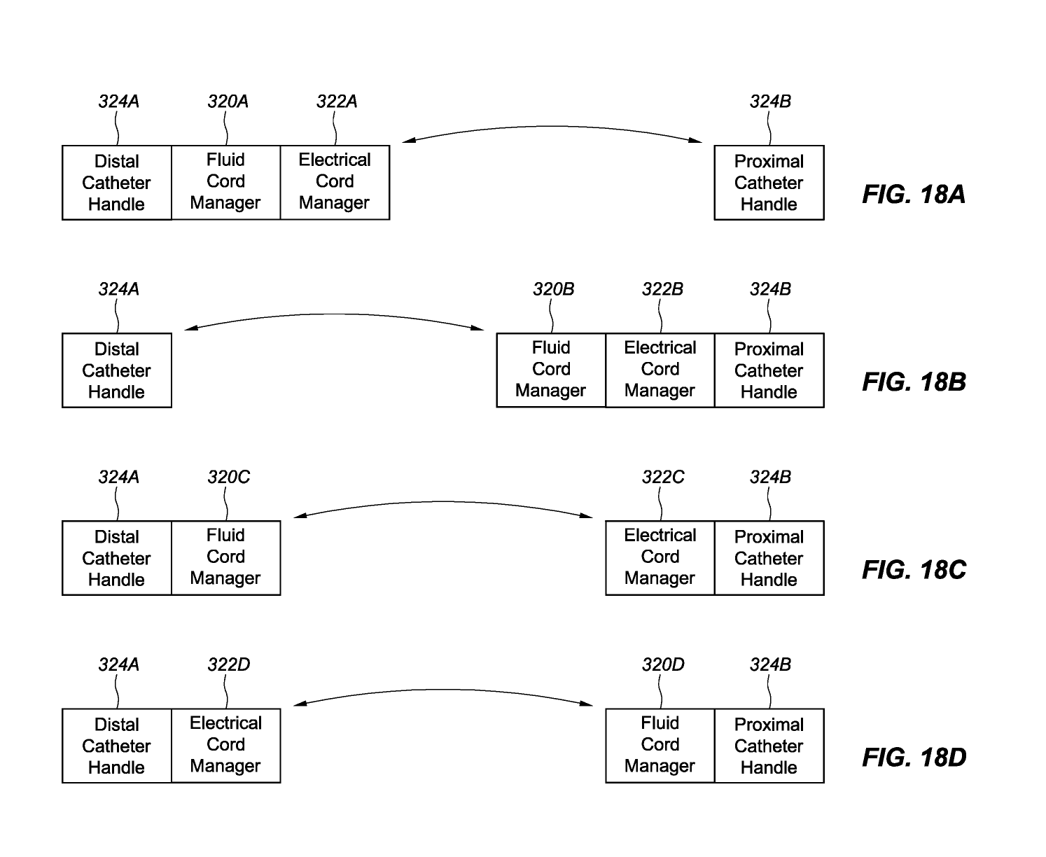

FIGS. 18A-18D provide high-level block diagrams illustrating various possible relationships between components of a cord management system and a catheter handle. In the illustrated embodiments, the cord management system includes two components—a fluid cord management system and an electrical cord management system. In FIG. 18A, a fluid cord manager 320A and an electrical cord manager 322A are attached to or comprise part of a distal end of a catheter handle 324A. The fluid cord manager 320A and the electrical cord manager 320B can be further configured to be connected (e.g., via a plurality of cords) to a proximal end of the catheter handle 324B. An example of the fluid cord manager 320A includes a free rotatory irrigation channel, as further described below. An example of the electrical cord manager 320B includes an electrical slip ring, configured to allow the transmission of power and electrical signals from stationary structure to a rotating structure, as discussed above.

As shown in FIGS. 18B, 18C, and 18D, other arrangements of the fluid and electrical cord management systems are possible. For example, as shown in FIG. 18B, both a fluid cord manager 320B and an electrical cord manager 322B can be attached to or comprise part of the proximal catheter handle 324B. As shown in FIG. 18C, the fluid cord manager 320C can be attached to or comprise part of the distal catheter handle 324A, while the electrical cord manager 322C can be attached to or comprise part of the proximal catheter handle 324A. The reverse situation, shown in FIG. 18D, allows the fluid cord manager 320D to be attached to or comprise part of the proximal catheter handle 324B, while the electrical cord manager 322D can be attached to or comprise part of the distal catheter handle 324A.

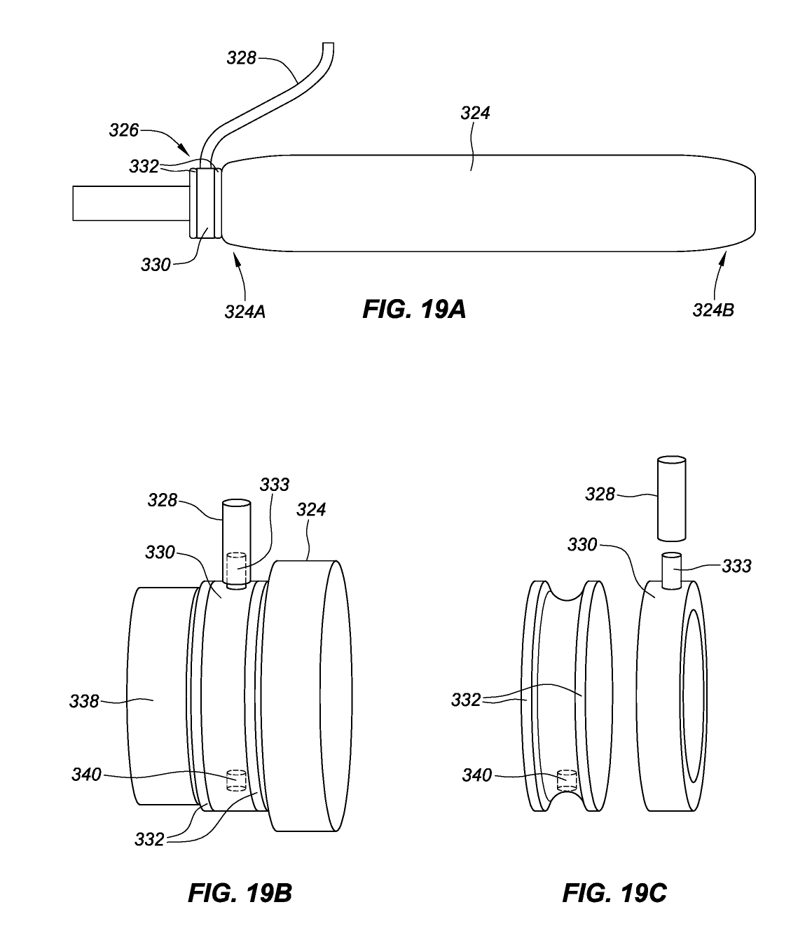

FIG. 19A illustrates an example of one type of fluid cord manager, a free rotatory irrigation channel 326. FIGS. 19B and 19C are zoomed and exploded views, respectively, of the free rotatory irrigation channel 326. As shown in FIG. 19A, the free rotatory irrigation channel 326 is located at the distal end 324A of the catheter handle 324 to minimize its size and interference with other control components in the handle 324. Nevertheless, the free rotatory irrigation channel 326 could be located at the proximal end 324B or elsewhere on the catheter handle 324.

The free rotatory irrigation channel 326 enables 360-degree free rotation of a catheter handle 324 without causing tangling of an associated irrigation tube 328 connecting the free rotatory irrigation channel 326 to a saline pump (not shown). The free rotatory irrigation channel 326 comprises a ring channel that is embedded in the handle 324 and can move freely with respect to the handle surface while maintaining continuous flow and seal of the irrigant. The ring channel includes two components: a free ring 330 and a fixed ring 332. The fixed ring 332 can be made of rubber or other elastic material, and the free ring 330 can be made from firm plastic material, for example. When the fixed ring 332 is assembled with the free ring 330, the fixed ring 332 can snuggle tightly around the free ring 330 and the two rings can form a tubular channel that seals the irrigant inside. The free ring 330 has a protruding cylindrical opening 333 on its outer surface that connects to the irrigation tube 328. The fixed ring 332 is fixed to the catheter handle 324, as shown in FIGS. 19A and 19B.

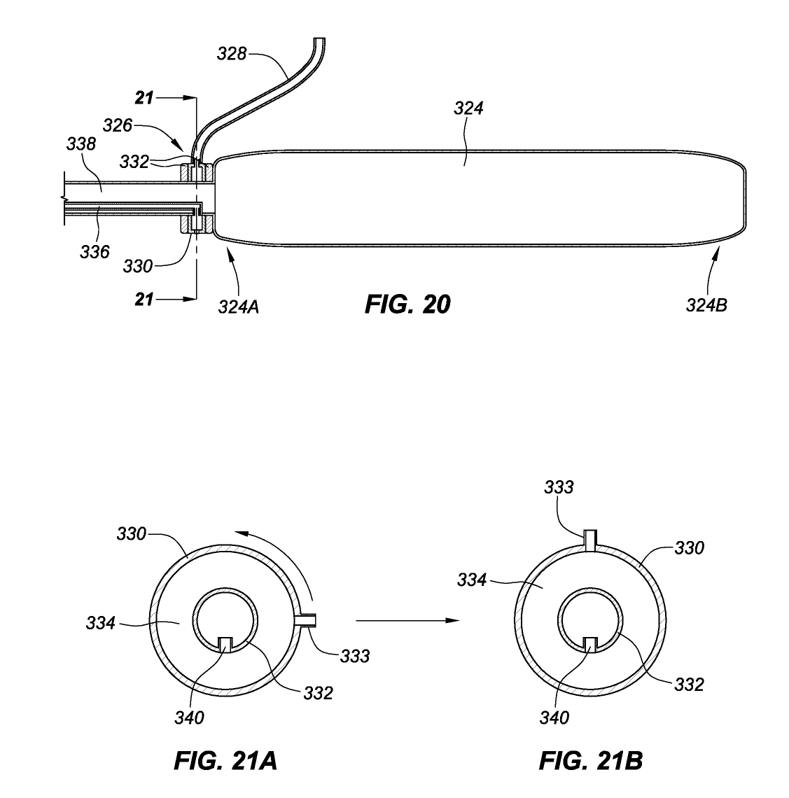

Fluid from the irrigation tube 328 is transferred, via the opening 333, into an irrigant chamber 334 in between the free ring 330 and the fixed ring 332, shown in FIGS. 20, 21A, and 21B. The catheter shaft tubing 336 conducts the irrigant through the catheter shaft 338 to the tip of the catheter (not shown). The fixed ring 332 also has a cylindrical opening 340, best shown in FIGS. 19A, 21A and 21B, that extends inwards and allows irrigant to enter the tubing 336 from the chamber 334.

As shown in FIGS. 21A and 21B, when the fixed ring 332 is assembled with the free ring 330, the free ring 330 rotates freely with respect to the fixed ring 332. When a user rotates the catheter handle 324, the fixed ring 332, which is fixed to the catheter handle 324, rotates with it. Because the free and fixed rings 330 and 332 can move freely with respect to each other, however, the free ring 330 can maintain its position (i.e., remain stationary) when the catheter handle 324 rotates. Thus, the irrigation tube 328 does not get tangled with the catheter handle 324 or any of its electric cables or other wires.

Although numerous embodiments of this invention have been described above with a certain degree of particularity, those skilled in the art could make numerous alterations to the disclosed embodiments without departing from the spirit or scope of this disclosure. All directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the any aspect of the disclosure. As used herein, the phrased “configured to,” “configured for,” and similar phrases indicate that the subject device, apparatus, or system is designed and/or constructed (e.g., through appropriate hardware, software, and/or components) to fulfill one or more specific object purposes, not that the subject device, apparatus, or system is merely capable of performing the object purpose. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A rotatable connector for connecting a catheter handle and a cable plug, the rotatable connector comprising:
a first support member configured to be stationary relative to the catheter handle and electrically coupled thereto when the catheter handle is connected to the first support member; and
a second support member configured to be stationary relative to the cable plug and electrically coupled thereto when the cable plug is connected to the second support member;
wherein the second support member is configured to rotate relative to the first support member when the catheter is rotated with respect to the cable plug.

2. The rotatable connector of claim 1, wherein the first support member comprises an outer cylinder and wherein the second support member comprises an inner cylinder with respect to the outer cylinder.

3. The rotatable connector of claim 1, further comprising a fluid rotary joint configured to allow transfer of fluid between the first support member and the second support member.

4. The rotatable connector of claim 3, further comprising a fluid lumen running through a central longitudinal axis of the rotatable connector.

5. The rotatable connector of claim 3, further comprising an electrical wire lumen running through a central longitudinal axis of the connector and a fluid lumen running parallel to the central longitudinal axis.

6. The rotatable connector of claim 5, further comprising a rotatable toroid element through which the fluid lumen runs, wherein the rotatable toroid element comprises a first half and a second half, and wherein the first and second halves are configured to rotate relative to one another.

7. The rotatable connector of claim 1, further comprising a servomechanism connected to the first and second support members, the servomechanism being configured to power rotation of the rotatable connector.

8. The rotatable connector of claim 7, wherein the servomechanism is configured to communicate with a motion processing unit used to determine a roll angle of the catheter handle.

9. The rotatable connector of claim 8, wherein the motion processing unit comprises at least one of an accelerometer, an inclinometer, or a gyroscope.

10. A rotatable connector for connecting a catheter handle to a cable plug, the rotatable connector comprising:
at least one of a slip ring and a fluid rotary joint, wherein the at least one of the slip ring and fluid rotary joint is located within at least one of the catheter handle and the cable plug, and wherein the cable plug is configured to be coupled to the catheter handle; and
a servomechanism configured to power rotation of the at least one of the slip ring and the fluid rotary joint.

11. The rotatable connector of claim 10, wherein the servomechanism is configured to communicate with a motion processing unit used to determine a roll angle of the catheter handle.

12. The rotatable connector of claim 11, wherein the motion processing unit comprises at least one of an accelerometer, an inclinometer, or a gyroscope.

13. The rotatable connector of claim 10, wherein the servomechanism is configured to communicate with a motion processing unit via a wireless connection.

14. The rotatable connector of claim 10, wherein the servomechanism comprises a servomotor.

15. A detangling device for a cable plug configured to connect to a catheter handle, the detangling device comprising:
a first support member comprising a first inner surface and a first outer surface, the first inner surface comprising a plurality of first electrical contacts, the first support member coupled to or located within the catheter handle and configured to be stationary relative to the catheter handle; and
a second support member comprising a second inner surface and a second outer surface, the second support member located within the first support member and configured to be stationary relative to the cable plug, wherein the second inner surface of the second support member comprises a plurality of second electrical contacts, each second electrical contact configured to electrically connect to at least one of the first electrical contacts;
wherein the cable plug is configured to be inserted into the second support member, and wherein the cable plug and a shaft tip of the catheter are configured to be electrically connected when the cable plug is fully inserted into the second support member; and
wherein the first support member is configured to rotate relative to the second support member.

16. The detangling device of claim 15, wherein the plurality of first electrical contacts comprises a plurality of band electrodes.

17. The detangling device of claim 15, wherein the plurality of second electrical contacts comprises at least one of a plurality of pin electrodes or a plurality of brushes.

18. The detangling device of claim 15, wherein the each of the plurality of first electrical contacts is configured to be electrically connected to at least one of a plurality of third electrical contacts located on an outer surface of the shaft tip of the catheter.

19. The detangling device of claim 18, wherein the plurality of third electrical contacts comprises a plurality of ring electrodes.

20. The detangling device of claim 15, wherein each of the plurality of second electrical contacts is configured to be electrically connected to one another and to a corresponding at least one of a plurality of fourth electrical contacts, the plurality of fourth electrical contacts being located on the second outer surface of the second support member.

21. The detangling device of claim 20, wherein the plurality of fourth electrical contacts comprises at least one of a plurality of pin electrodes or a plurality of brushes.

22. The detangling device of claim 20, wherein each of a plurality of fifth electrical contacts located on an outer surface of the cable plug is configured to be electrically connected to at least one of the plurality of third electrical contacts, when the cable plug is fully inserted into the second support member.

23. The detangling device of claim 15, further comprising a locking mechanism configured to inhibit rotation of the first support member relative to the second support member when the locking mechanism is engaged.

24. The detangling device of claim 23, wherein the locking mechanism comprises an engagement/disengagement system based on at least one of the following: a mechanical force, a frictional force, or a pneumatic force.

25. The detangling device of claim 15, wherein the first support member comprises an outer cylinder and wherein the second support member comprises an inner cylinder.

26. A cord management system configured to manage a cord connection between a catheter handle and at least one of an energy supply or a fluid supply, the cord management system comprising:
a means for inhibiting cord entanglement; and
a means for cord detangling.

27. A rotatable connector for connecting a catheter handle and an irrigation tube, the rotatable connector comprising:
a first support member configured to be stationary relative to the catheter handle; and
a second support member configured to be stationary relative to the irrigation tube;
wherein the second support member is configured to rotate relative to the first support member when the catheter handle is rotated with respect to the irrigation tube.

28. The rotatable connector of claim 27, wherein the first support member comprises a fixed cylinder and wherein the second support member comprises a free cylinder with respect to the fixed cylinder.

29. The rotatable connector of claim 28, wherein the fixed cylinder is configured to snug tightly around the free cylinder and seal an irrigant inside the rotatable connector.
